(19) United States
(12) Patent Application Publication
(10) Pub. No.: US 2015/0071860 A1
(43) Pub. Date: Mar. 12, 2015

(54) CYCLIC PEG COATINGS INCREASE THE BLOOD CIRCULATION TIME OF PARTICLES USED IN DRUG DELIVERY APPLICATIONS

(71) Applicants: Coleen Pugh, Akron, OH (US); Gladys R. Montenegro-Galindo, Akron, OH (US)

(72) Inventors: Coleen Pugh, Akron, OH (US); Gladys R. Montenegro-Galindo, Akron, OH (US)

(21) Appl. No.: 14/481,389

(22) Filed: Sep. 9, 2014

Related U.S. Application Data
(60) Provisional application No. 61/875,134, filed on Sep. 9, 2013.

(51) Int. Cl. A61K 9/107; C08G 65/34; A61K 47/48; A61K 47/34; A61K 49/00

(52) U.S. Cl. CPC A61K 9/1075; A61K 47/34; A61K 49/0082; A61K 47/48215; C08G 65/34
USPC 424/9.6; 424/497; 514/772.1; 424/9.1; 525/418; 428/402

(57) ABSTRACT

A micelle comprising a diblock polymer with a cyclic polyethylene glycol block and a hydrophobic block is provided. The micelle may be used in conjunction with one or a combination of therapeutic agents, targeting agents, and imaging agents. The micelle may be incorporated in a pharmaceutical composition.

### CROSS-REFERENCE TO RELATED APPLICATIONS

This application claims priority from U.S. Provisional Patent Application No. 61/875,134 filed on Sep. 9, 2013, the contents of which are incorporated herein by reference.

### FIELD OF THE INVENTION

One or more embodiments relate to diblock polymers with a cyclic polyethylene glycol block and a hydrophobic block, micelles prepared from di-block polymers with a cyclic polyethylene glycol block and a hydrophobic block, and methods of treating a disease comprising of administering a therapeutic amount of a micelle comprising a diblock polymer with a cyclic polyethylene glycol block and a hydrophobic block.

### BACKGROUND OF THE INVENTION

The goals of drug delivery devices are to deploy the bioactive compound to a target site, reduce side effects, prevent drugs from degrading, maintain therapeutic levels for prolonged periods of time, to have predictable and controllable release rates, reduce dosing frequency, increase patient compliance, and reducing drug costs by improving the cost-effectiveness of the treatment.

Controlled drug delivery prevents side effects from toxic drug levels and provides a more consistent drug dose over time. In a typical injection, the drug is administered at or close to the toxic drug level and the drug concentration quickly depletes to the sub-therapeutic drug level, and then to zero, at which time a new injection is required. In contrast, when a controlled release-drug carrier is injected the drug concentration increases until it reaches a point in the therapeutic window, and the concentration remains in the therapeutic window for a prolonged period of time. Controlled release also helps improve patient compliance due to the fact that the drug does not need to be administered as frequently as an injected drug solution.

In order to achieve a desired sustained release profile, the drug should remain in the body long enough to achieve a therapeutic effect. To reduce premature elimination, devices should be small in size (e.g <150 nm). Devices should also be coated with a hydrophilic layer to reduce premature elimination. Thus, there is a need in the art to produce a polymer for use in a device with some or all of these features.

### SUMMARY OF THE INVENTION

A first embodiment provides a micelle comprising a diblock polymer with a cyclic polyethylene glycol block and a hydrophobic block.

A second embodiment provides a micelle as in the first embodiment, where the diblock polymer is a cyclic-polyethylene glycol-block-polyester.

A third embodiment provides a micelle as in the either the first or second embodiment, where the diblock polymer is defined by formula 1:

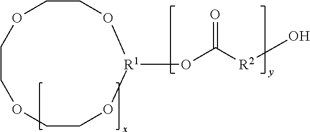

where x is about 5 to about 19 units, y is about 4 to about 30 units, R¹ is a trivalent organic group, and each R² is independently a divalent organic group.

A forth embodiment provides a micelle as in any of the first through third embodiments, where the diblock polymer is defined by formula 2:

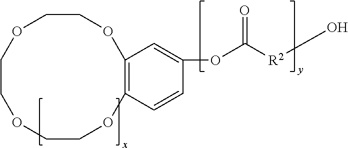

where x is about 5 to about 19 units, y is about 4 to about 30 units, each R² is independently a divalent organic group.

A fifth embodiment provides a micelle as in any of the first through forth embodiments, where the diblock polymer is defined by formula 3:

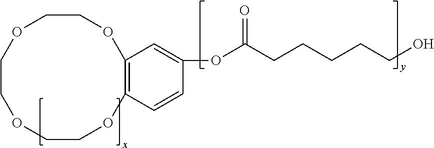

where x is about 5 to about 19 units and y is about 4 to about 30 units.

A sixth embodiment provides a micelle as in any of the first through fifth embodiments, where the diblock polymer is defined by formula 4:

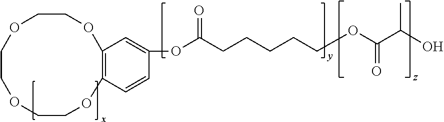

where x is about 5 to about 19 units, y is about 0 to about 30 units, and z is about 1 to about 25 units.

A seventh embodiment provides a micelle as in any of the first through sixth embodiments, where the micelle has a diameter of about 30 nm to about 450 nm.

An eighth embodiment provides a micelle as in any of the first through seventh embodiments, where the micelle further includes an imaging agent.

A ninth embodiment provides a micelle as in any of the first through eighth embodiments, where the imaging agent is selected from the group consisting of fluorophores, biotin derivatives, and quantum dots.

A tenth embodiment provides a micelle as in any of the first through ninth embodiments, where the imaging agent is coupled to a diblock polymer with a polyethylene glycol block and a hydrophobic block.

An eleventh embodiment provides a micelle as in any of the first through tenth embodiments, where the micelle further includes a targeting agent.

A twelfth embodiment provides a micelle as in any of the first through eleventh embodiments, where the targeting agent is selected from the group consisting of folic acid, epidermal growth factor receptor (EGFR), vascular endothelial growth factor (VEGF), vascular-targeting agent (VTA).

A thirteenth embodiment provides a micelle as in any of the first through twelfth embodiments, where the targeting agent is coupled to a diblock polymer with a polyethylene glycol block and a hydrophobic block.

A fourteenth embodiment provides a micelle as in any of the first through thirteenth embodiments, where the micelle encloses a therapeutic agent.

A fifteenth embodiment provides a micelle as in any of the first through fourteenth embodiments, where the therapeutic agent encapsulated or covalently bound to the hydrophobic component of the micelle.

A sixteenth embodiment provides a pharmaceutical composition comprising the micelle of claim 1 and a pharmaceutically acceptable excipient.

A seventeenth embodiment provides a diblock polymer defined by formula 1:

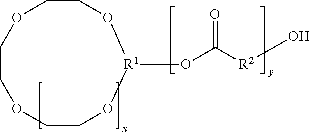

where x is about 5 to about 19 units, y is about 4 to about 30 units, R¹ is a trivalent organic group, and each R² is independently a bivalent organic group.

An eighteenth embodiment provides a di-block polymer of the seventeenth embodiment, defined by formula 2:

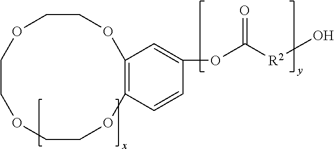

where x is about 5 to about 19 units, y is about 4 to about 30 units, each R² is independently a bivalent organic group.

A nineteenth embodiment provides a di-block polymer of the seventeenth embodiment or eighteenth embodiment defined by formula 3:

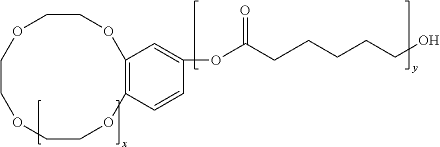

where x is about 5 to about 19 units and y is about 4 to about 30 units.

A twentieth embodiment provides a di-block polymer of the any the seventeenth embodiment through nineteenth embodiments defined by formula 4:

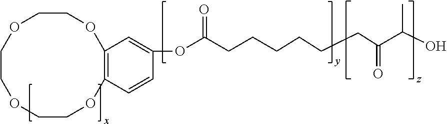

where x is about 5 to about 19 units, y is about 0 to about 30 units, and z is about 1 to about 25 units.

A twenty-first embodiment provides a method of treating a disease comprising of administering a therapeutic amount of a micelle comprising a diblock polymer with a cyclic polyethylene glycol block and a hydrophobic block, where the micelle encloses a therapeutic agent.

### BRIEF DESCRIPTION OF THE DRAWINGS

FIG. 1 provides a chart of the comparison of the drug release profiles of injection vs. controlled release systems.

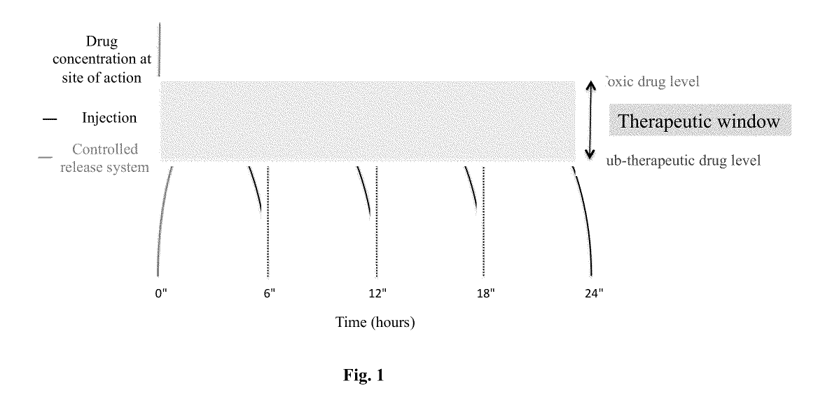

FIG. 2 provides a scheme showing a comparison of micelles formed by the cyclic-b-linear and linear-b-linear PEG-b-PCL diblock copolymers and their micelles presented in this study.

FIG. 3 provides a matrix-assisted laser desorption/ionization time-of-flight mass spectra of MCBnOH with potassium ions (trifluoroacetate salt).

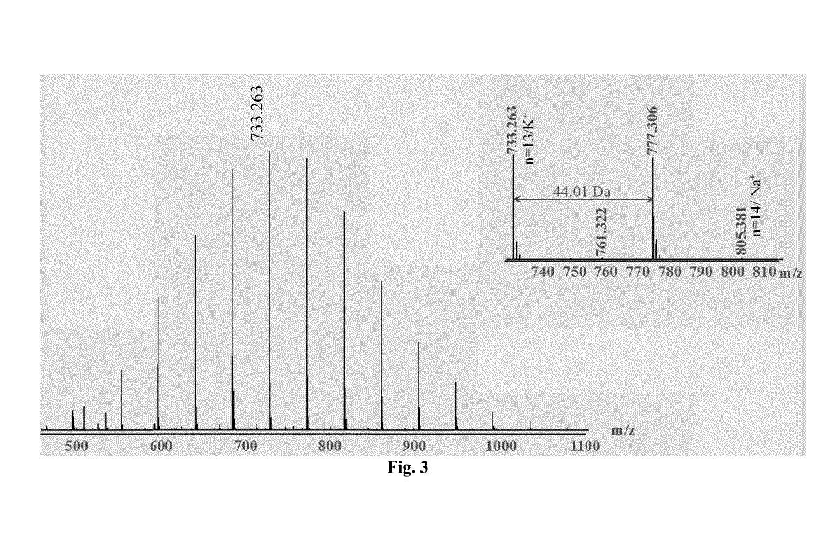

FIG. 4 provides matrix-assisted laser desorption/ionization time-of-flight mass spectra of MeOPEG550BnOH with potassium ions (trifluoroacetate salt).

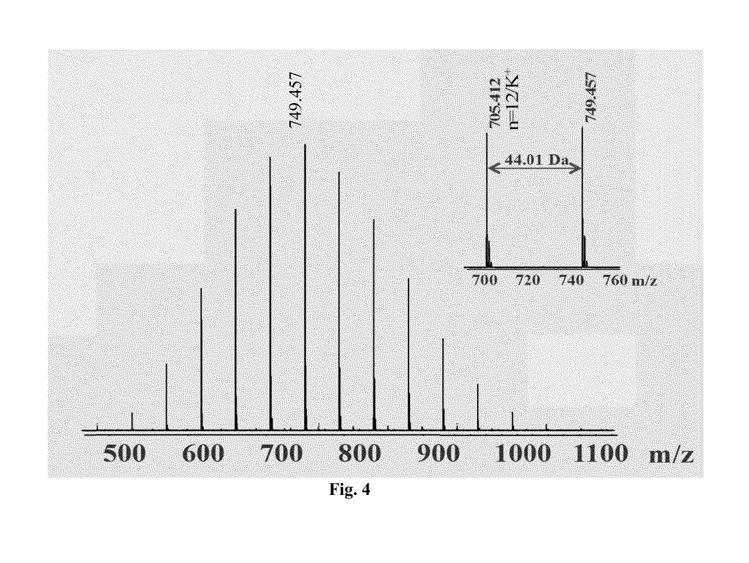

FIG. 5A provides a 1H NMR Spectra of MCBnOPCL.

FIG. 5B provides 1H NMR Spectra of MCBnOH.

FIG. 6 provides a Gel Permeation Chromatogram of the MCBnOH Macroinitiator and Diblock PEG-b-PCL Copolymers with PCL Mn corresponding to 1×, 3×, and 5× the Molecular Weight of the PEG Block.

FIG. 7 provides a Gel Permeation Chromatogram of the MeOPEG550BnOH Macroinitiator and PEG-b-PCL Diblock Copolymers with PCL Mn corresponding to 1×, 3×, and 5× the Molecular Weight of the PEG Block.

FIG. 8A provides a chart of the results of Differential Scanning calorimetry (DSC) of Cyclic-b-Linear PEG-b-PCL Diblock Copolymers8ADifferential Scanning calorimetry (DSC) of Cyclic-b-Linear PEG-b-PCL Diblock Copolymers

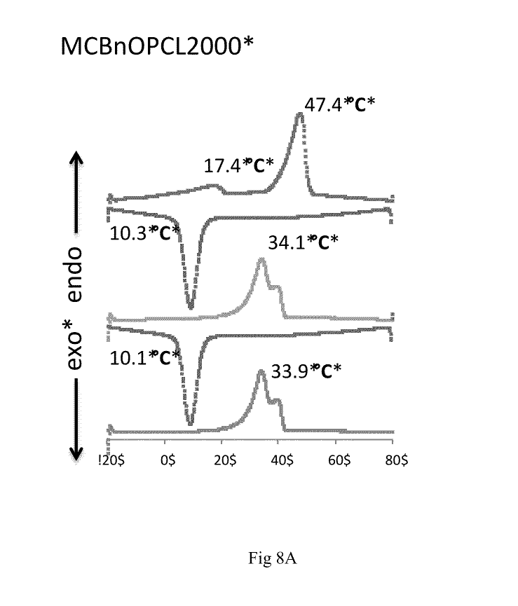

FIG. 8B provides a chart of the results of Differential Scanning calorimetry (DSC) of Cyclic-b-Linear PEG-b-PCL Diblock Copolymers

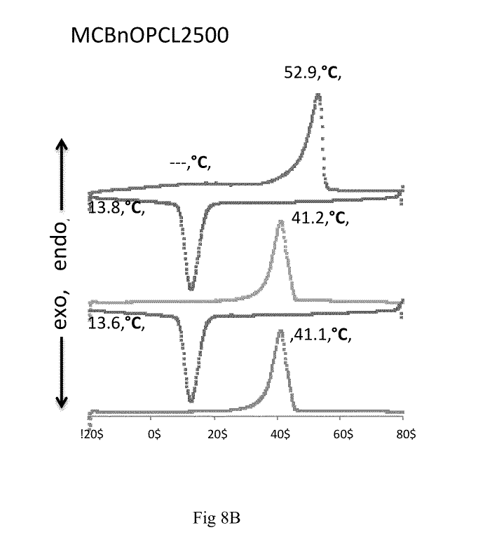

FIG. 9A provides a chart of the results of Differential Scanning calorimetry (DSC) of Cyclic-b-Linear PEG-b-PCL Diblock Copolymers

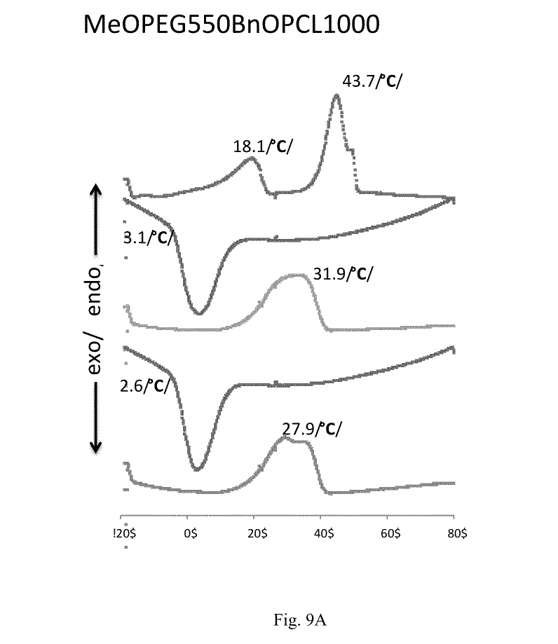

FIG. 9B provides a chart of the results of Differential Scanning calorimetry (DSC) of Cyclic-b-Linear PEG-b-PCL Diblock Copolymers

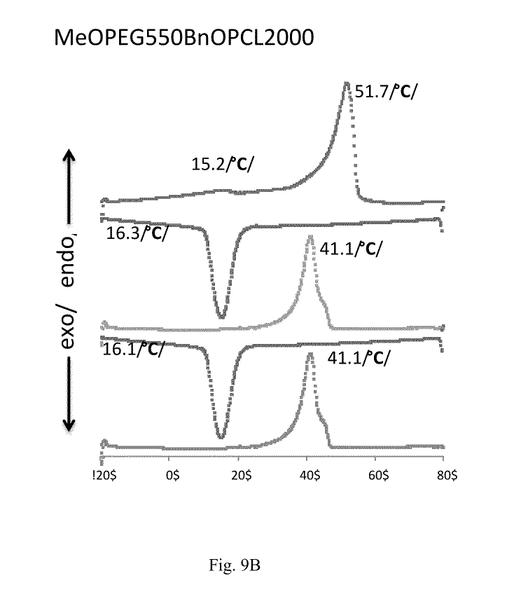

### DETAILED DESCRIPTION OF ILLUSTRATIVE EMBODIMENTS

One or more embodiments are directed to a micelle comprising a diblock polymer with a cyclic polyethylene glycol block and a hydrophobic block. The micelle comprising a diblock polymer with a cyclic polyethylene glycol block and a hydrophobic block, or “device,” advantageously has been found to have an architecture that decreases protein adsorption on the surface of the device. Decreased protein adsorption reduces the elimination of the device and provides increased circulation time. The device may include a function incorporated or encompassed by the micelle. Suitable functions include the addition of a targeting agent or an imaging agent. The device may also encompasses a therapeutic agent.

The diblock polymer with a cyclic polyethylene glycol block and a hydrophobic block may be referred to herein as the diblock polymer. The diblock polymer includes a cyclic polyethylene glycol block attached to a hydrophobic block by a bond or a linking group.

The cyclic polyethylene glycol block has the structure of a crown ether with repeating ethyleneoxy units and a location for the attachment of a hydrophobic block. In one or more embodiments, the cyclic polyethylene glycol block has about 7 to about 21 repeating ethyleneoxy units, in other embodiments about 8 to about 14 repeating ethyleneoxy units, and in still other embodiments about 10 to about 12 repeating ethyleneoxy units.

In one or more embodiments, the cyclic polyethylene glycol block may be defined by the formula:

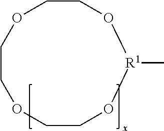

where x is about 5 to about 19 units and R¹ is a trivalent organic group.

In one or more embodiments, the hydrophobic block includes about 4 to about 30 repeating units, in other embodiments, about 20 to about 30 repeating units, and in still other embodiments, about 28 to about 30 repeating units. Suitable polymers for use in the hydrophobic block include, but are not limited to polyesters, polyanhydrides, and other biodegradable hydrophobic polymers. Specific examples of polyesters include polycaprolactone and poly(lactic acid) [poly(glycolic acid)].

In one or more embodiments, the diblock polymer with a cyclic polyethylene glycol block and a hydrophobic block may be a cyclic-polyethylene glycol-block-polyester. In these or other embodiments, the diblock polymer may be defined by formula 1:

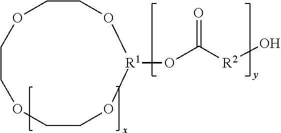

where x is about 5 to about 19 units, y is about 4 to about 30 units, R¹ is a trivalent organic group, and each R² is independently a divalent organic group.

Suitable trivalent organic groups for use as linking groups in a diblock polymer include linear, branched, cyclic, and aromatic organic groups with three valences. The trivalent organic group may include about 1 (or the minimum required carbons to prepare a group) to 30 carbon atoms. In one or more embodiments, the trivalent organic groups may also include heteroatoms.

In one more embodiments, where the linking group, R′, of formula 1 is a trivalent benzene group, the diblock polymer may be defined by formula 2:

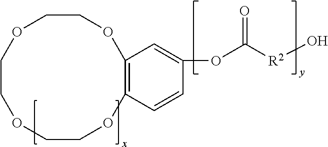

where x is about 5 to about 19 units, y is about 4 to about 30 units, each R² is independently a divalent organic group.

In one or more embodiments, where each y unit is a caprolactone unit, the diblock polymer may be defined by formula 3:

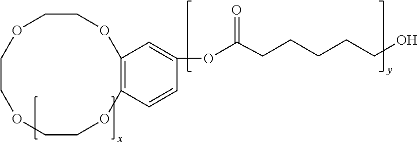

where x is about 5 to about 19 units and y is about 4 to about 30 units.

In one or more embodiments, where the each y unit is a poly(lactic acid) unit and/or caprolactone unit, the diblock polymer may be defined by formula 4:

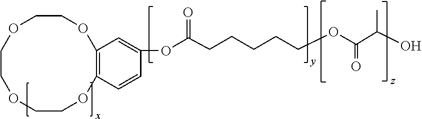

where x is about 5 to about 19 units, y is about 0 to about 30 units, and z is about 1 to about 25 units. In one or more embodiments, the sum of the x and y units of a diblock polymer defined by formula 4 is less than or equal to 30 total units.

The diblock polymers may self assemble into micelles depending on different characteristics of the micellar system and process variables. When the concentration of block copolymer exceeds the critical micelle concentration (CMC) in an aqueous medium the hydrophobic blocks form the core of the micelle, while the hydrophilic block forms the corona.

In one or more embodiments, micelles may be prepared from the diblock polymers by a co-solvent evaporation method. In these or other embodiments, the micelles may be prepared by combining the diblock polymer with a water miscible solvent to prepare a diblock polymer solution, combing the diblock polymer solution with an aqueous medium, and removing the water miscible solvent. Suitable methods for removing the water miscible solvent include evaporation or dialysis. A co-solvent evaporation method is described in Aliabadi, et al., Encapsulation of Hydrophobic Drugs in Polymeric Micelles Through Co-Solvent Evaporation: the Effect of Solvent Composition on Micellar Properties and Drug Loading. Int. J. Pharm. 2007, 329, 158-165, which is incorporated herein by reference.

In one or more embodiments, a therapeutic agent or an imaging agent may be entrapped in the core during self-assembly. The therapeutic agent or imaging agent may be hydrophobic to allow it to interact with the hydrophobic end of the diblock polymer. The therapeutic agent or imaging agent may also be covalently bound to the hydrophobic block of a diblock polymer.

A volume to volume ratio may be used to characterize the amount of water miscible solvent and aqueous medium used to prepare a micelle from a diblock polymer. In one or more embodiments, the volume to volume ratio of the water miscible solvent to the aqueous medium is about 50:1 to 500:1, in other embodiments about 100:1 to about 300:1, and in still other embodiments about 150:1 to about 200:1.

A weight to weight ratio may be used to characterize the amount of diblock polymer combined with the water miscible solvent. In one or more embodiments, the weight to weight ratio of the diblock polymer to the water miscible solvent is about 0.001:1 to 0.1:1, in other embodiments about 0.005:1 to about 0.05:1, and in still other embodiments about 0.01:1 to about 0.035:1.

In one or more embodiments, water miscible solvents are organic solvents that form a homogenous solution when mixed with an aqueous medium. Suitable water miscible solvents for use in a co-solvent evaporation method for producing micelles include low boiling point solvents (bp<70° C.), which favor removal while keeping micelle structure intact. Examples of suitable water miscible solvents for use in a co-solvent evaporation method for producing micelles include, but are not limited to, acetone and tetrahydrofuran (THF).

In one or more embodiments, an aqueous medium is water or a solution where water is the solvent. Suitable aqueous mediums for use in a co-solvent evaporation method for producing micelles include buffered aqueous solutions or therapeutic aqueous solution, which include, but are not limited to, phosphate buffered saline, ultra-pure (type 1) water, and water for injection.

In one or more embodiments, micelles may be prepared from the diblock polymers by a direct dissolution method. In the direct dissolution method, a solid sample of the diblock polymer is directly dissolved in the aqueous medium to prepare a micellar system. The micellar system may be annealed by standing or by thermal treatment, usually through ultrasonic agitation. Optionally, the copolymer and water are mixed at high temperatures to favor micellization.

Micelle size may be adjusted by controlling the ratio of the water miscible solvent to the aqueous medium, length of hydrophilic block, length of hydrophobic block, ratio of hydrophilic/hydrophobic polymer, amount of diblock polymer, and/or ratio of solvent evaporation. The size of the micelle may be measured dynamic light scattering. In one or more embodiments, the micelle has a diameter of about 30 nm to about 450 nm, in other embodiments about 30 nm to about 200 nm, and in still other embodiments about 30 nm to about 50 nm.

In one or more embodiments, the size of the micelle may be selected to cause the device to accumulate at a tumor site. In these or other embodiments, micelles loaded with therapeutic agents for cancer treatment or therapy can accumulate at the tumor site due to the enhanced permeability and retention (EPR) effect. The EPR effect is a result of rapid blood vessel growth at the site of the tumor, which causes leaky vessels, or more precisely, defects in the endothelial lining of the new blood vessels. Leaky vessels permit particles of certain sizes to permeate into the cancerous tissue. Thus, by increasing the blood circulation time of micelles coupled with an increased concentration of blood vessels and blood vessel defects can provide more effective cancer treatment. In these or other embodiments, the micelle has a diameter of about 40 nm to about 60 nm.

As noted above, one or more embodiments may include a function incorporated or encompassed by the micelle. Suitable functions include the addition of a targeting agent or an imagine agent.

In one or more embodiments, a targeting agent may be a ligand incorporated on the surface of the micelle. Micelles with one or more targeting ligands located on the surface of the micelle may be utilized to selectively target tumor cells, as well as other maladies that produces receptor proteins and can complex with targeting ligands. The increased circulation time of micelles with targeting functionality are advantageous, because of the increased probability of binding to receptors with a single dose.

Suitable targeting agents include, but are not limited to, folic acid, epidermal growth factor receptor (EGFR), vascular endothelial growth factor (VEGF), vascular-targeting agent (VTA), and other cancer related targeting agents.

In one or more embodiment, the targeting agent may be coupled to a polymer. In these or other embodiments, the polymer-coupled targeting agent may be included with the diblock polymer during formation of the micelle to prepare a micelle with a targeting agent incorporated on the micelle's surface. In one or more embodiment, the polymer-coupled targeting agent includes a targeting agent portion and a hydrophobic, polymeric portion. In these or other embodiments, the polymer-coupled targeting agent includes a targeting agent portion, a hydrophobic, polymeric portion, and a hydrophilic, polymeric portion. In certain embodiments, the targeting agent is coupled to a diblock polymer with a polyethylene glycol block and a hydrophobic block.

In one or more embodiments, the device may include an imaging agent. Imaging agents may be useful, for example, in for diagnostic purposes. Devices with imaging agents may optionally include targeting agents. In one or more embodiments, devices with imagining agents may accumulate in solid tumors via the EPR with or without the assistance of a targeting agents. In one or more embodiments, the imaging agent is selected from the group consisting of fluorophores, biotin derivatives, and quantum dots.

In one or more embodiments, imaging agents may be encompassed by the micelle. In these or other embodiments, an imaging agents may be incorporated on the surface of the micelle.

In one or more embodiment, the imaging agent may be coupled to a polymer. In these or other embodiments, the polymer-coupled imaging agent may be included with the diblock polymer during formation of the micelle to prepare a micelle with an imagining agent incorporated on the micelle's surface. In one or more embodiment, the polymer-coupled imaging agent includes an imaging agent portion and a hydrophobic, polymeric portion. In these or other embodiments, the polymer-coupled imaging agent includes an imaging agent portion, a hydrophobic, polymeric portion, and a hydrophilic, polymeric portion. In certain embodiments, the imaging agent is coupled to a diblock polymer with a polyethylene glycol block and a hydrophobic block.

In one or more embodiments, the micelle encapsulates a therapeutic agent. The device with an encapsulated a therapeutic agent may optionally be utilized with a targeting agent, imaging agent, or both. Suitable therapeutic agents include gene therapy agents and other therapeutic bioactive compounds. Example of a therapeutic agent is doxorubicin alkylating agents such as mechlorethamine, antimetabolites such as methotrexate, anti-microtubule agents such as paclitaxel, topoisomerase inhibitors such as camptothecin, and cytotoxic antibiotics such as actinomycin.

A therapeutic amount of bioactive substance will be determined by one skilled in the art as that amount necessary to effect treatment of a particular disease or condition, taking into account a variety of factors such as the patient's weight, age, and general health, the therapeutic properties of the drug, and the nature and severity of the disease. Typically, a therapeutic amount drug of a drug is an amount that when administered produces a concentration within the therapeutic window. Though, one skilled in the art may determine that different amount is necessary. The therapeutic window has an upper limit of the toxic drug level and a lower limit of the sub-therapeutic drug level. Advantageously, when the device includes an encapsulated therapeutic agent, the concentration of the therapeutic agent may be maintained in the therapeutic window due to the increased circulation time of the device.

In one or more embodiments, the device may be included in a pharmaceutical composition. Pharmaceutical compositions may include the device and at least one pharmaceutically acceptable excipient. Pharmaceutically acceptable excipients include natural or synthetic substance formulated alongside the device for the purpose of bulking formations, diluting formulations, conferring therapeutic enhancement, or for ease in manufacturing.

While the device may be particularly useful in pharmaceutical compositions intravenous, where architecture of the device advantageously decreases protein adsorption, the device may also find use in other types of pharmaceutical compositions. The pharmaceutical compositions include those suitable for subdermal, inhalation, oral, topical or parenteral use. Examples of pharmaceutical compositions include, but are not limited to, tablets, capsules, powders, granules, lozenges, or liquid preparations.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monoleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Pharmaceutical compositions may contain from about 0.1% to about 70% by weight of the device depending on the method of administration. In these or other embodiments, pharmaceutical composition may include about 0.001% to about 30% of a pharmaceutical agent.

While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

### Materials

PEG 600 (Sigma-Aldrich) and PEG550 were azeotropically dried from toluene solutions prior to use, triethylamine (Sigma-Aldrich, 99%) was dried from and stored in KOH, sodium borohydride (Sigma-Aldrich, 98%) was stored in the drybox and used as received, ε-caprolactone (Acros Organics, 99%) was dried from CaH₂ prior to use. Methanesulfonyl chloride (Aldrich, 99.5%), 3,4 dihydroxybenzaldehyde (Acros Organics, 97%), potassium carbonate (Sigma-Aldrich, 99%), ethanol (Pharmco-Aaper, 200 proof absolute alcohol), 4-hydroxybenzyl alcohol (Sigma-Aldrich, 99%), tin octoate (Aldrich. 94%), NaCl (Sigma-Aldrich, 99%), KCl (BHD, 99%), Na₂HPO₄ (Fisher, 99.9%), and KH₂PO₄ (Fisher, 99.7%) were used as received. Acetone was filtered 3× through a 0.45 μm Scientific Tisch PTFE membrane filter, the block copolymers used for self-assembly were diluted in acetone then filtered 3× through the same 0.45 μm PTFE filters. Phosphate Buffer Saline solution (PBS) was prepared according to standard recipe from MilliQ Water and NaCl, KCl, Na₂HPO₄, and KH₂PO₄. PBS and MilliQ Water used for self-assembly were filtered 3× through 0.45 μm ThermoScientific polyethersulfone membrane filters. Reagent grade tetrahydrofuran (THF) was dried by distillation from purple sodium benzophenone ketyl under N₂. All other reagents and solvents were commercially available and used as received.

Methods.

All reactions were performed under a N₂ atmosphere using a Schlenk line or a vacuum atmospheres drybox, unless noted otherwise. For self-assembly experiments, the solvents used were filtered 3× through a 0.45 μm PTFE filter from Scientific Tisch, the polymers were dissolved in acetone and the solutions filtered 3×, the acetone was removed under vacuum to yield filtered polymer. The vials, tubes and bottles used were rinsed with filtered solvents. Silica gel (Sorbent Technologies, 63-200 mm mesh size, 60 Å pore size) was used for column chromatography. ¹H NMR spectra (δ, ppm) were recorded on either a Varian Mercury 300 (300 MHz and 75 MHz, respectively), an INOVA 500 (500 MHz and MHz respectively), or an INOVA 750 (750 MHz and 188 MHz, respectively) spectrometer. All spectra were recorded in CDCl₃ or a mixture of CDCl₃ and DMSO-d₆ unless mentioned otherwise; and the resonances were measured relative to the residual solvent resonances and referenced to tetramethylsilane (0.00 ppm). Number-(Mₙ) and weight average (M_{w}) molecular weights relative to linear polystyrene (GPCPst) and polydispersities (PDI=M_{w}/Mₙ) were determined by gel permeation chromatography (GPC) from calibration curves of log Mₙ vs. elution volume at 35° C. using THF as solvent (1.0 mL/min), a set of 50 Å, 100 Å, 500 Å, 10⁴ Å, and linear (50-10⁴ Å) Styragel 5 μm columns, a Waters 486 tunable UV/Vis detector set at 254 nm, a Waters 410 differential refractometer, and Millenium Empower 2 software. All samples (approximately 0.5 g/L) were dissolved overnight and filtered through a 0.45 μm PTFE filter. Micellar sizes and polydispersities were measured by Dynamic Light Scattering using a Brookhaven Instrument coupled with a BI-200SM goniometer, BI-9000AT correlator, and an EMI-9863 photomultiplier tube for photon counting. A Meller Griot 35 mW He—Ne laser was used as light source (632.8 nm). The software BIC DLS software version 3.40 was used to obtain the hydrodynamic radius of PEG-b-PCL micelles. A cylindrical glass scattering cell with a diameter of 12 mm was placed at the center of the thermostated bath with decahydronaphthalene used for refractive index matching. For each sample, three separate DLS measurements were taken at 25° C. Each DLS measurement was taken for 10 min at a 90° angle with a wavelength of 639 nm. The data was analyzed in terms of volume-weighted and number-weighted distributions.

A Perkin-Elmer Pyris 1 differential scanning calorimeter was used to determine T_{g} values, which were read as the middle of the change in heat capacity. The reported T_{g} values of the polymer and copolymers are the mean values from the second and third heating scans. All heating and cooling rates were 10° C./min. Transition temperatures were calibrated using indium and zinc, tin, or benzophenone standards. Matrix-assisted laser desorption ionization time of flight mass spectrometry (MALDI ToF-MS) experiments were carried out on a Bruker Ultraflex III TOF/TOF mass spectrometer (Bruker Daltonics, Inc., Billarica, Mass.), equipped with a Nd:YAG laser emitting at a wavelength of 355 nm. All spectra were measured in positive reflector mode. The instrument was calibrated prior to each measurement with an external PMMA standard. The preparation of the samples analyzed involved the following steps. Tetrahydrofuran (THF) solutions of the matrix, t-2-(3-(4-t-Butyl-phenyl)-2-methyl-2-propenylidene) malononitrile (DCTB, 20 mg/mL) and the cationizing agent, potassium-trifluoroacetic acid (KTFA, 10 mg/mL) in THF were mixed in 10:1 ratio, and 0.5 μL, of the final mixture were deposited on microliter plate wells (MTP 384-well ground steel plate). After the spots were dried, 0.5 μL, of a solution of the polymer in CHCl₃ (10 mg/mL) were deposited on top of the matrix and salt layer. Micelle pictures were taken using a transmission electron microscope (TEM Philips TECNAI) with an accelerating voltage of 120 kV. To observe micelle morphologies, a drop of the original micelle solution was placed on a Formvar/carbon-coated grid. The excess solution was blotted away with a Kimwipe. The grids were dried at room temperature and atmospheric pressure for several hours before examination in the TEM.

### Synthesis of 3,4-(42-Crown-14)benzyl Alcohol (MC-BnOH)

MC-BnOH was synthesized as described previously (see Pugh, C.; Bae, J. Y.; Scott, J. R.; Wilkins, C. L. Amphiphilic Approach for Preparing Homopolyrotaxanes of Poly(Ethylene Oxide). Macromolecules 1997, 30, 8139-8152 incorporated herein by reference), except that we used acetonitrile as a solvent and established a new procedure for removing dimers and trimers from 3,4-(42-crown-14)benzaldehyde (MC-CHO), before proceeding to subsequent steps. We will therefore reproduce only the synthesis of 3,4-(42-crown-14)-benzaldehyde (MC-CHO) and report its purification here. A solution of 3,4-dihydroxybenzaldehyde (0.50 g, 3.6 mmol) and PEG-600 bismesylate (2.7 g, 3.6 mmol) in acetonitrile (125 mL) was added dropwise over 13 h to a slurry of finely ground K₂CO₃ (0.75 g, 5.4 mmol) in acetonitrile (25 mL) at 82° C. The temperature was raised to 87° C. and the mixture was stirred for 72 h. After the reaction mixture was cooled down to room temperature, it was filtered through a fritted glass funnel to remove the potassium salts. Acetonitrile was removed by rotary evaporation to obtain 2.7 g (102%) of a brown oil. This brown oil contains dimeric and trimeric impurities and was purified by flash chromatography using 3:1 acetone:hexanes to obtain 1.0 g (39%) of high purity MC-CHO as a yellow oil; Mₙ=0.815*10³ g/mole, PDI=1.05. ¹H-NMR (500 MHz): 3.64-3.77 (m, —OCH₂—), 3.92 (dd, —CH₂CH₂OAr, ²J=8.5 Hz, ³J=3.9 Hz), 4.23 (dt, —CH2OAr, ²J=14.4 Hz, ³J=4.8 Hz), 6.99 (dd, 1 aromatic H meta to —CH₂OAr, ²J=10.2 Hz, ³J=4.9 Hz), 7.43 (dd, 1 aromatic H ortho to —CHO, ortho and meta to —CH₂OAr and 1 aromatic H ortho to —CHO, meta and para to —CH₂OAr, ²J=13.7 Hz, ³J=6.5 Hz), 9.83 (s, —CHO). ¹³C NMR (125 MHz): 70.93 (—OCH₂—), 112.25 (1 aromatic C metha to —CHO, ortho to —CH₂OAr), 112.84 (1 aromatic C ortho to —CHO, ortho to —CH₂OAr), 126.92 (1 aromatic C ortho to —CHO, metha to —CH₂OAr), 130.59 (1 aromatic C substituted with CHO), 191.13 (C═O). The purity of the MC-CHO unimer was confirmed using MALDI-ToF MS. The reduction of MC-CHO to MC-BnOH proceeded as described previously.³³

### Synthesis of α-Methyl Ether, ω-Benzyl Alcohol Polyethylene Glycol 550 (MeOPEG550BnOH)

MeOPEG550BnOH was synthesized in 64% yield from α-methyl ether, ω-mesylated poly(ethylene glycol) 550 (MeOPEG550Ms). MeOPEG550Ms was synthesized as by Soltero, R.; Radhakrishan, B.; Ekwuribe, N. N.; Rehlaender, B.; Hickey, A.; Bovet, L. L. Pharmaceutical Compositions of Insulin Drug-Oligomer Conjugates and Methods of Treating Diseases Therewith. WO03022208, 2005, incorporated herein by reference. In a typical example, MeOPEG550Ms (9.0 g, 14 mmol), K₂CO₃ (2.9 g, 21 mmol), potassium iodide (93 mg, 0.56 mmol), and 4-hydroxybenzyl alcohol (3.0 g, 24 mmol) were dissolved in acetonitrile (90 mL). After stirring at room temperature for 5 min, the reaction was immersed in an oil bath at 90° C. After 48 h of reaction, the reaction was cooled down, the precipitated salts were filtered out using a frit, the acetonitrile was rotary evaporated to obtain 6.0 g (64%) of a clear oil. Mₙ=1.78*10³ g/mole, PDI=1.13. 1H-NMR (300 MHz): 3.38 (s, CH3-), 3.65 (m, —OCH2-), 3.86 (t, —CH2CH2-OAr—, J=4.9 Hz), 4.13 (t, —CH2CH2-OAr—, J=4.9 Hz), 4.62 (s, —CH2OH), 6.91 (ArC(2,6)H), 7.30 (ArC(3,5)H).

### Synthesis of MC-BnO_PCL Diblock Copolymers (cyclicPEG-b-linearPCL)

CyclicPEG-b-linearPCL diblock copolymers were synthesized in 36-70% yield as in the following example. In a Schlenk tube weighed MC-BnOH (0.0711 g, 0.1027 mmol) followed by ε-caprolactone (0.0664 g, 0.5817 mmol) and a stock solution of tin octoate (0.0498 g, 0.1229 mmol) in dry THF. Freeze-pump-thawed 2× (10-30-20), and immersed in oil bath at 80° C. for 19 h. Rotavapped THF until the solution was viscous. Precipitated 3× in 60 mL of hexanes.

### Synthesis of MeOPEG550BnO-PCL Diblock Copolymers (linearPEG-b-linearPCL)

LinearPEG-b-linearPCL diblock copolymers were synthesized in 29-54% yield as in the following example. In a Schlenk tube weighed MeOPEG550BnOH (0.0450 g, 0.0818 mmol), followed by ε-caprolactone (0.1902 g, 1.6664 mmol) and a stock solution of tin octoate (0.0333 g, 0.0823 mmol) in dry THF. Freeze-pump-thawed 2× (10 min-30 min-20 min), and immersed in oil bath at 80° C. for 19 h. Rotavapped THF until the solution was viscous. Precipitated 3× in 60 mL of hexanes.

### Self-Assembly of PEG-b-PCL Diblock Copolymers

Self-assembled micelles using PEG-b-PCL were prepared by modifying a literature procedure. In a typical example, cyclicPEG-b-PCL (2.5 mg) is dissolved in 0.1000 g of acetone. Added 15 mL of PBS or MilliQ water at 0.1 mL/min. Covered vial with a kimwipe and stirred at normal temperature and pressure for 2.5 h to let acetone slowly evaporate. After that, transferred to a Bell Jar using the aspirator vacuum overnight.

### Results and Discussion Section

### Synthesis, Purification, and MALDI-ToF MS Characterization of MC-BnOH

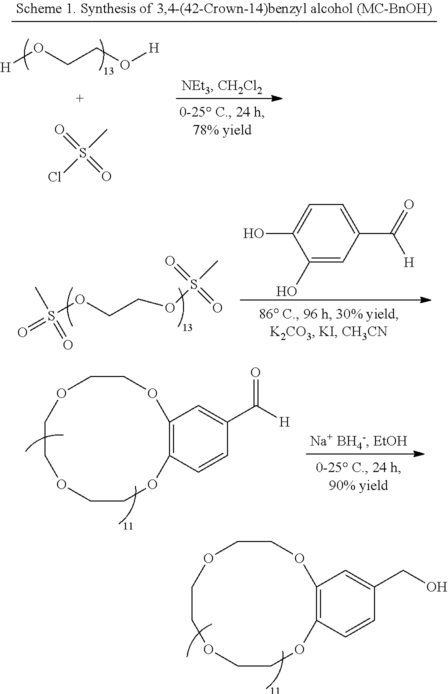

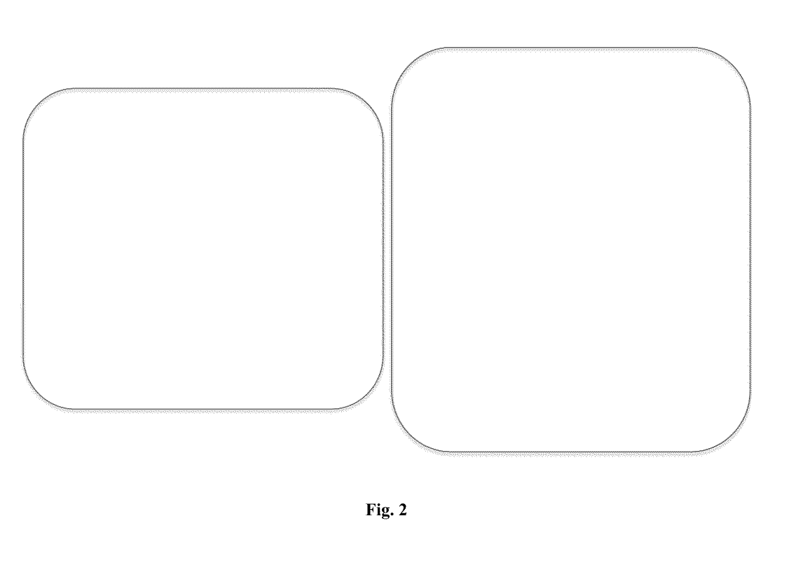

Although we previously synthesized MCBnOH in relatively high yield by the route outlined in Scheme 1, we thought it would be possible to increase the yield by using an alternative technique for the separation of unimers from dimers to trimers. We have therefore repeated this synthesis and confirmed that the product of each step was pure by MALDI-ToF MS before proceeding to the next step. As said previously, the most important step in the synthesis is the macrocyclization of PEG600-bismesylate with 3,4 dihydroxybenzaldehyde in the presence of multiple potassium template ions. In the 2005 paper a new route for the purification involved obtaining pure macrocyclic unimer in 19.4% the purification step to get rid of larger cycles involving Soxhlet extraction would reduce the yield significantly. As an alternative, we developed a method using only column chromatography to remove the larger cycles. We increased the yields of cyclic unimer from 19.4% to 30% to give an overall yield of 21% vs. 8-16% using Soxhlet extraction. In FIG. 2 the results corresponding to the Macromolecules 2005 article are compared with the results from this article.

### Synthesis and MALDI-ToF Characterization of MeOPEG550BnOH

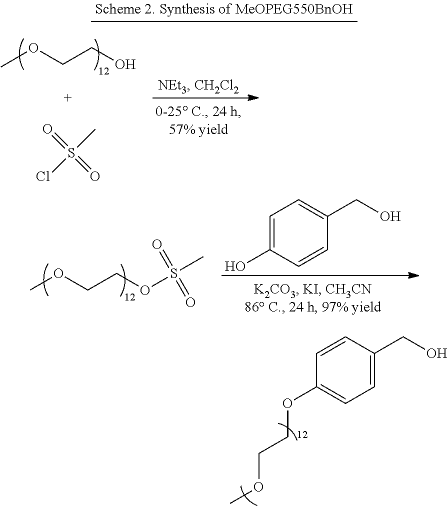

Scheme 2 outlines the synthesis of MeOPEG550BnOH starting from MeOPEG550OH which was first converted to the corresponding methanesulfonate ester according to literature procedure. The next step is an etherification to yield an α-methoxy, ω̅ benzyl-alcohol PEG. This was followed by reduction of the benzaldehyde to benzyl alcohol. MeOPEG550Ms was then etherified using an excess of 4-hydroxybenzyl alcohol and slightly substoichiometric amounts of K₂CO₃ to 4-hydroxybenzyl alcohol to prevent deprotonation of the benzyl alcohol that would lead to coupling between PEG chains.

### Synthesis and Characterization of MCBnOPCL Diblock Copolymers

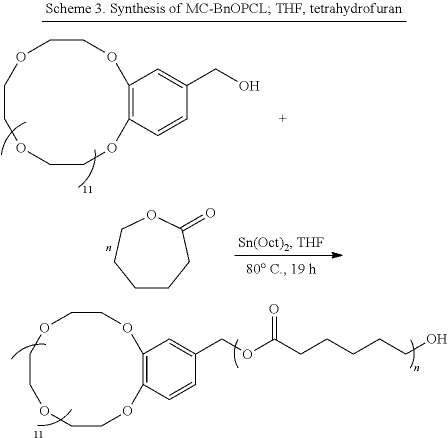

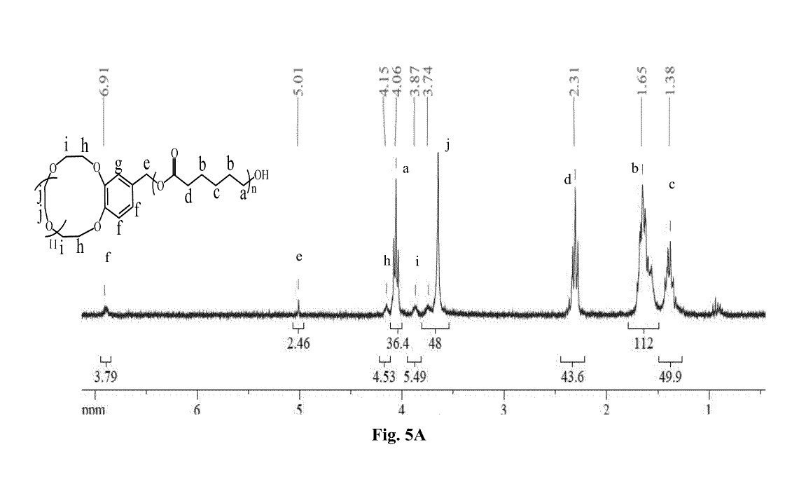

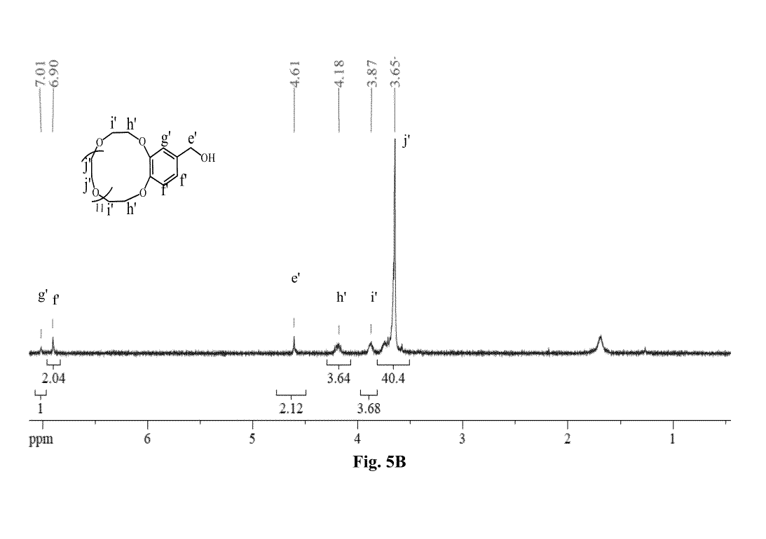

Scheme 3 shows the synthesis of MCBnOPCL (i.e. cyclic-b-linear) diblock copolymer by ring-opening polymerization of ε-caprolactone using tin octoate as a catalyst and THF as solvent. MCBnOH as macroinitiator FIGS. 5A and 5B the stacked ¹H NMR spectra of the MCBnOH macroinitiator and the MCBnOPCL diblock copolymer. All the resonances in MCBnOH remain the same in the diblock copolymer except for the benzyl alcohol methylene that shifts from 4.61 ppm to 5.01 in the diblock copolymer. Maldi-ToF also confirms the synthesis of the MCBnOPCL diblock copolymer. The zoomed in region between 1355 and 1443 Da has the assignments for the number of ethylene glycol and caprolactone units in the potassiated diblock copolymers. For example, MC₁₀BnOPCL₇K⁺ has a mass of 1399.8 Da.

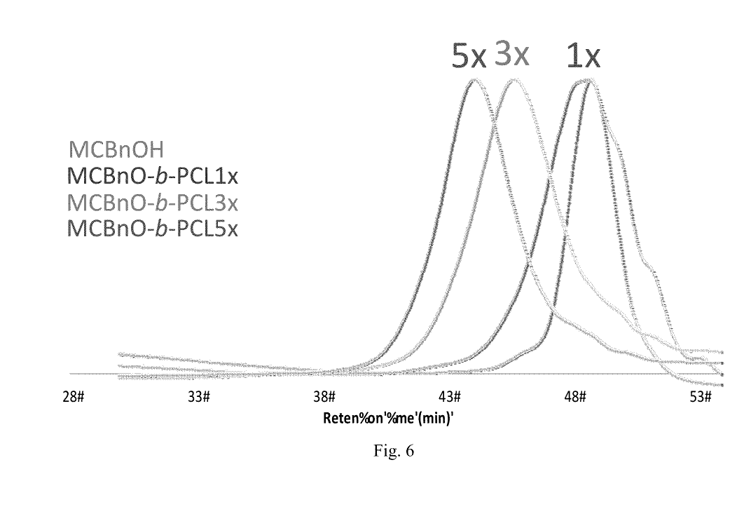

FIG. 6 shows the Gel Permeation Chromatography traces for MCBnOH and three different PEG-b-PCL diblock copolymers with PCL molecular weights corresponding to one time, three times, and five times the molecular weight of the PEG block. The traces show the gradual reduction in retention times corresponding to the increase of molecular weight. Finally, Table 1 summarizes the reaction conditions and molecular weights obtained for the MCBnOPCL and MeOPEG550BnOPCL diblock copolymers discussed in the next section. For the diblock copolymer syntheses the ratio between the initiator (i.e. PEG either MCBnOH or MeOPEG550BnOH) and tin octoate was kept close to 1 and the conversions were close to 100%, obtaining polymers with polydispersities in the range between 1.03 to 1.23.

The published thermal transitions for polycaprolactone homopolymers with a molecular weight of 8000 g/gmole are Tg at −60° C., melting temperature at 55° C., and crystallization temperature at 33° C. In the case of PEG homopolymer with a molecular weight of 4000 g/gmole, Tg is at −22° C., melting temperature at 52° C., and crystallization temperature at 38° C. Differential scanning calorimetry of the cyclic-b-linear PEG-b-PCL diblock copolymers between −20 and 80° C. should only show melting and crystallization transitions because PEG with a molecular weight lower than 1000 Da does not crystallize. In the case of MCBnO-b-PCL2000, polymorphic structures are seen in the second and third heating scans with melting temperatures around 34° C. In contrast, MCBnO-b-PCL2500 does not show polymorphic behavior and the melting transitions are at 41° C. The melting transitions increase with the molecular weight of polycaprolactone, as expected.

TABLE 1
Synthesis of PEG-b-PCL Diblock Copolymers with either Cyclic or Linear
Architecture

| | | | | Theo PCL Mn | | Theo PCL Mn | PCL Mn | Mn (GPC) | |
|---|---|---|---|---|---|---|---|---|---|
| | Initiator | [M]/[I] | [I]/[Sn(Oct)₂] | (kg/mol) | Conv (%) | with conversion | (¹H NMR) | (kg/mol) | PDI |
| Linear | MeOPEG550BnOH | 4 | 1.0 | 0.44 | 100.0 | 0.4 | 0.4 | 1.56 | 1.03 |
| | MeOPEG550BnOH | 12 | 1.1 | 1.32 | 96.0 | 1.3 | 1.1 | 2.01 | 1.09 |
| | MeOPEG550BnOH | 20 | 1.0 | 2.32 | 98.8 | 2.3 | 1.9 | 2.54 | 1.20 |
| Cyclic | MC-BnOH | 6 | 0.8 | 0.65 | 92.9 | 0.6 | 0.7 | 1.76 | 1.12 |
| | MC-BnOH | 15 | 0.8 | 1.77 | 96.1 | 1.7 | 1.7 | 2.31 | 1.17 |
| | MC-BnOH | 24 | 0.8 | 2.70 | 91.7 | 2.5 | 2.5 | 3.14 | 1.23 |

### Synthesis and Characterization of MeOPEG550BnOPCL Diblock Copolymers

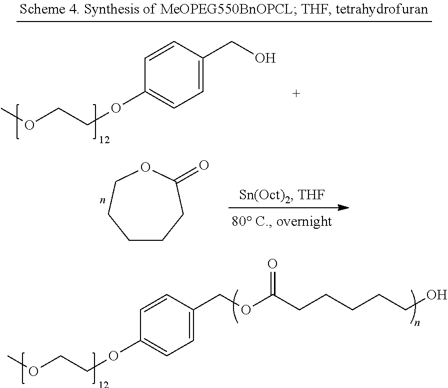

We have synthesized linear-b-linear diblock copolymers as a control to compare the effect of the PEG architecture on micelle self-assembly. These linear-b-linear diblock copolymers were synthesized in an analogous way to MCBnOPCL: by ring-opening polymerization of ε-caprolactone using MeOPEG550BnOH as macroinitiator, tin octoate as catalyst, and THF as solvent.

¹H NMR was performed on MeOPEG550BnOH and MeOPEG550BnOPCL. As in the case of the cyclic-b-linear diblock copolymers, all the MeOPEG550BnOH remain the same in the diblock copolymer except for the benzyl alcohol methylene that shifts from 4.62 to 5.01 ppm.

Likewise, Maldi-ToF confirms the synthesis of the diblock copolymer. The Maldi-ToF spectrum shows between 1363 and 1441 Da some of the repeating units for each block. For example, MeOPEG₁₀PCL₇K⁺ has a mass of 1415.8 Da.

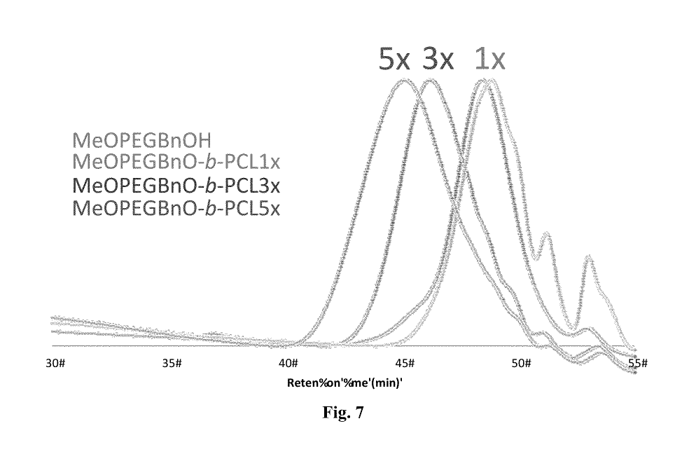

In FIG. 7, Gel Permeation Chromatography traces show the gradual increment of molecular weight from MeOPEG550BnOH for diblock copolymers with PCL molecular weights that correspond to one time, three times, and five times the molecular weight of the PEG block.

Similar DSC thermograms were obtained for MeOPEG550-b-PCL diblock copolymers. In the case of MeOPEG550-b-PCL1000, polymorphic structures are also seen in the second and third heating scans with melting temperatures around 28° C. In contrast, MeOPEG550-b-PCL2000 show less of a polymorphic behavior and the melting transitions are at 41° C., similar to the case of MCBnO-b-PCL2500. The melting transitions increase with the molecular weight of polycaprolactone. MeOPEG550-b-PCL polymers seem to crystallize better than MCBnO-b-PCL.

### Self-assembly and Characterization of PEG-b-PCL Micelles

Micelles were prepared by diblock copolymer self-assembly by solvent evaporation using acetone as solvent and water or PBS as selective solvent. The micelle sizes were between 39-1326 nm by dynamic light scattering, as seen on Table 2. However, transmission electron microscopy images showed that all micelles were under 100 nm and the larger sizes were due to micelle aggregation. The aggregation was more important in the case of Phosphate Buffer Saline (PBS) as compared to deionized MilliQ Water. This might be due to enhanced interaction of micelles with the salts in the PBS medium with PEG, which in turn enhanced micelle-to-micelle interaction.

TABLE 2
Micelle Sizes by Dynamic Light Scattering

| | |
|---|---|
| MilliQ Water | PBS |

| Sample | Size (nm) | Error (nm) | Size (nm) | Error (nm) |
|---|---|---|---|---|
| L1x | 39 | 5 | 207 | 22 |
| L3x | 164 | 9 | 181 | 14 |
| L5x | 83 | 9 | 48 | 6 |
| C1x | 33 | 4 | 201 | 24 |
| C3x | 200 | 41 | 1326 | 44 |
| C5x | 89 | 0 | 439 | 26 |

### CONCLUSIONS

PEG-b-PCL diblock copolymers with linear or cyclic architectures in the PEG block (i.e. MeOPEG550-b-PCL vs. MCBnO-b-PCL) and different molecular weights in the PCL block were synthesized from PEG macroinitiators. The PCL molecular weights corresponded to one time, three times, and five times the molecular weight of the PEG macroinitiator. These diblock copolymers were characterized using NMR spectroscopy, MALDI-ToF, Gel Permeation Chromatography, and Differential Scanning calorimetry. Polymers with low polydispersity (i.e. 1.03 to 1.23) were obtained. DSC between −20° C. and 80° C. showed only glass transition temperatures between 28 to 41° C. corresponding to the PCL block. Self-assembly of the amphiphilic diblock copolymers using a solvent evaporation method in PBS or water led to micelles characterized using Dynamic Light Scattering and Transmission Electron Microscopy. Micellar sizes were between 39 to 200 nm. Aggregation was observed especially in PBS for the MCBnO-b-PCL diblock copolymers. The synthesis and self-assembly of these diblock copolymers will enable a PEG architecture relevant in drug delivery applications.

What is claimed is:

1. A micelle comprising a diblock polymer with a cyclic polyethylene glycol block and a hydrophobic block.

2. The micelle of claim 1, where the diblock polymer is a cyclic-polyethylene glycol-block-polyester.

3. The micelle of claim 2, where the diblock polymer is defined by formula 1: 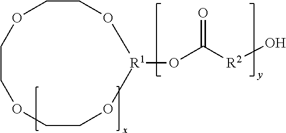
where x is about 5 to about 19 units, y is about 4 to about 30 units, R¹ is a trivalent organic group, and each R² is independently a divalent organic group.

4. The micelle of claim 3, where the diblock polymer is defined by formula 2: 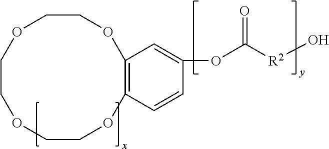
where x is about 5 to about 19 units, y is about 4 to about 30 units, each R² is independently a divalent organic group.

5. The micelle of claim 3, where the diblock polymer is defined by formula 3: 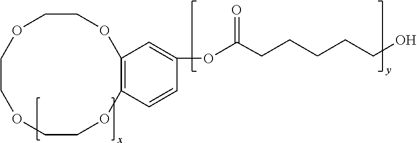
where x is about 5 to about 19 units and y is about 4 to about 30 units.

6. The micelle of claim 3, where the diblock polymer is defined by formula 4: 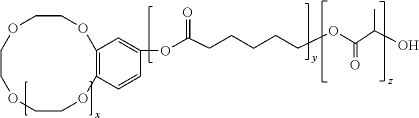
where x is about 5 to about 19 units, y is about 0 to about 30 units, and z is about 1 to about 25 units.

7. The micelle of claim 1, where the micelle has a diameter of about 30 nm to about 450 nm.

8. The micelle of claim 1, where the micelle further includes an imaging agent.

9. The micelle of claim 8, where the imaging agent is selected from the group consisting of fluorophores, biotin derivatives, and quantum dots.

10. The micelle of claim 8, where the imaging agent is coupled to a diblock polymer with a polyethylene glycol block and a hydrophobic block.

11. The micelle of claim 1, where the micelle further includes a targeting agent.

12. The micelle of claim 11, where the targeting agent is selected from the group consisting of folic acid, epidermal growth factor receptor (EGFR), vascular endothelial growth factor (VEGF), vascular-targeting agent (VTA).

13. The micelle of claim 11, where the targeting agent is coupled to a diblock polymer with a polyethylene glycol block and a hydrophobic block.

14. The micelle of claim 1, where the micelle encloses a therapeutic agent.

15. The micelle of claim 14, where the therapeutic agent encapsulated or covalently bound to the hydrophobic component of the micelle.

16. A pharmaceutical composition comprising the micelle of claim 1 and a pharmaceutically acceptable excipient.

17. A diblock polymer defined by formula 1: 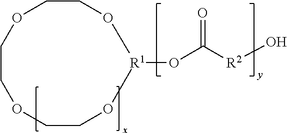
where x is about 5 to about 19 units, y is about 4 to about 30 units, R¹ is a trivalent organic group, and each R² is independently a bivalent organic group.

18. The diblock polymer of claim 17, defined by formula 2: 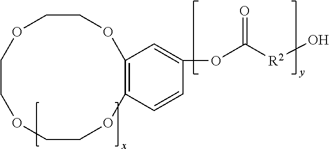
where x is about 5 to about 19 units, y is about 4 to about 30 units, each R² is independently a bivalent organic group.

19. The diblock polymer of claim 17, defined by formula 3: 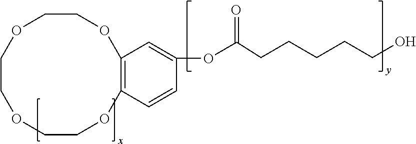
where x is about 5 to about 19 units and y is about 4 to about 30 units.

20. The diblock polymer of claim 17, defined by formula 4: 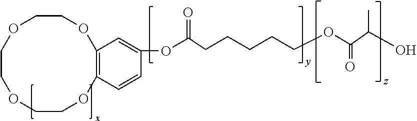
where x is about 5 to about 19 units, y is about 0 to about 30 units, and z is about 1 to about 25 units.

21. A method of treating a disease comprising of administering a therapeutic amount of a micelle comprising a diblock polymer with a cyclic polyethylene glycol block and a hydrophobic block, where the micelle encloses a therapeutic agent.
